(19) United States
(12) Patent Application Publication
(10) Pub. No.: US 2023/0277864 A1
(43) Pub. Date: Sep. 7, 2023

(54) THERAPEUTIC LIGHT IRRADIATION UNIT, THERAPEUTIC LIGHT IRRADIATION APPARATUS, AND ENDOSCOPE

(71) Applicant: OLYMPUS CORPORATION, Tokyo (JP)

(72) Inventor: Ryo MACHIDA, Kanagawa (JP)

(73) Assignee: OLYMPUS CORPORATION, Tokyo (JP)

(21) Appl. No.: 18/115,181

(22) Filed: Feb. 28, 2023

Related U.S. Application Data
(60) Provisional application No. 63/315,177, filed on Mar. 1, 2022.

(51) Int. Cl. A61N 5/06

(52) U.S. Cl. CPC A61N 5/0603; A61N 2005/0666

(57) ABSTRACT

A therapeutic light irradiation device including a reflecting surface disposed on a path of a therapeutic light emitted in a first direction from an insertion section of an endoscope, and configured to switch an irradiation direction of the therapeutic light between the first direction and a second direction intersecting the first direction, and a connector configured to detachably connect the reflecting surface to the insertion section.

### CROSS REFERENCE TO RELATED APPLICATIONS

This application claims the benefit of U.S. Provisional Application No. 63/315,177, filed Mar. 1, 2022, which is hereby incorporated by reference in its entirety.

### TECHNICAL FIELD

The present disclosure relates to a therapeutic light irradiation unit, a therapeutic light irradiation apparatus, and an endoscope.

### BACKGROUND ART

Light therapy includes technique of accumulating photosensitive drugs in tumors and irradiating them with therapeutic light for treatment, such as PDT (photo-dynamic-therapy) and PIT (photo-immuno-therapy).

Light therapy inside the body under endoscopic observation involves difficulty in capturing treatment sites at a position on the display screen where the therapeutic light is readily radiated due to effects of blurring caused by patients' pulsations or the like. In particular, it is difficult to capture treatment sites that cannot be viewed from the front with an endoscope, such as tumors on the inner walls of gastrointestinal tracts, in the center of the display screen. This makes it difficult to reliably irradiate the treatment sites with the therapeutic light.

On the other hand, since tumors can occur in various sites inside the bodies, it is desirable to be able to irradiate affected areas both on the front side and lateral side with therapeutic light without changing the endoscope. Therefore, a light therapy apparatus capable of selecting an irradiation region of therapeutic light has been proposed (see, for example, PTL 1). An apparatus described in PTL 1 can change the irradiation position of the therapeutic light emitted from a window of a second sheath by rotating or longitudinally moving a second opaque sheath within a first translucent sheath.

### SUMMARY

One aspect of the present disclosure is a therapeutic light irradiation device comprising a reflecting surface disposed on a path of a therapeutic light emitted in a first direction from an insertion section of an endoscope, and configured to switch an irradiation direction of the therapeutic light between the first direction and a second direction intersecting the first direction, and a connector configured to detachably connect the reflecting surface to the insertion section.

Another aspect of the present disclosure is a therapeutic light irradiation apparatus comprising an insertion section, a light source configured to radiate light in a first direction from the insertion section, and a reflecting surface disposed on a path of the light emitted in the first direction from the insertion section, and configured to switch an irradiation direction of the light between the first direction and a second direction intersecting the first direction.

Yet another aspect of the present disclosure is an endoscope comprising an insertion section, an illumination light source, arranged in the insertion section, configured to radiate illumination light in a first direction, a therapeutic light source, arranged in the insertion section, configured to radiate therapeutic light in the first direction, a reflecting surface disposed on a path of the illumination light and the therapeutic light emitted in the first direction from the insertion section, and configured to switch irradiation directions of both the illumination light and the therapeutic light between the first direction and a second direction intersecting the first direction, and a connector configured to detachably connect the reflecting surface to the insertion section.

### BRIEF DESCRIPTION OF DRAWINGS

FIG. 1

FIG. 1 is a configuration diagram of a therapeutic light irradiation unit and a therapeutic light irradiation apparatus according to a first configuration example of one embodiment of the present disclosure. {FIG. 2A}

FIG. 2A is a configuration diagram of a therapeutic light irradiation unit and a therapeutic light irradiation apparatus according to a second configuration example of one embodiment of the present disclosure. {FIG. 2B}

FIG. 2B is a configuration diagram of the therapeutic light irradiation unit and the therapeutic light irradiation apparatus according to the second configuration example of the one embodiment of the present disclosure.

{FIG. 3A}

FIG. 3A is a configuration diagram of a therapeutic light irradiation unit of FIGS. 2A and 2B, further including a wire. {FIG. 3B}

FIG. 3B is a configuration diagram of the therapeutic light irradiation unit of FIGS. 2A and 2B further including a wire. {FIG. 4A}

FIG. 4A is a configuration diagram of a modification of the therapeutic light irradiation unit of FIGS. 3A and 3B. {FIG. 4B}

FIG. 4B is a configuration diagram of the modification of the therapeutic light irradiation unit of FIGS. 3A and 3B. {FIG. 5A}

FIG. 5A is a configuration diagram of a modification of the therapeutic light irradiation unit of the second configuration example. {FIG. 5B}

FIG. 5B is a configuration diagram of the modification of the therapeutic light irradiation unit of the second configuration example.

FIG. 6

FIG. 6 is a configuration diagram of a therapeutic light irradiation unit and a therapeutic light irradiation apparatus according to a third configuration example of one embodiment of the present disclosure.

FIG. 7

FIG. 7 is a diagram showing spectral transmission characteristics of a reflecting member in the therapeutic light irradiation unit of FIG. 6. {FIG. 8A}

FIG. 8A is a configuration diagram of a modification of a therapeutic light irradiation unit further including a plurality of positioning portions. {FIG. 8B}

FIG. 8B is a configuration diagram of the modification of the therapeutic light irradiation unit further including a plurality of positioning portions. {FIG. 9A}

FIG. 9A is a diagram showing a fixture for fixing the therapeutic light irradiation unit to an insertion section. {FIG. 9B}

FIG. 9B is a cross-sectional view of the fixture and the insertion section.

FIG. 10

FIG. 10 is a configuration diagram of a therapeutic light irradiation system according to one embodiment of the present disclosure.

FIG. 11

FIG. 11 is a configuration diagram of a therapeutic light irradiation unit having a sheath-type connection section and a therapeutic light irradiation system including the unit.

### DESCRIPTION OF EMBODIMENTS

A therapeutic light irradiation unit, a therapeutic light irradiation apparatus, and an endoscope according to one embodiment of the present disclosure is to be described with reference to the drawings.

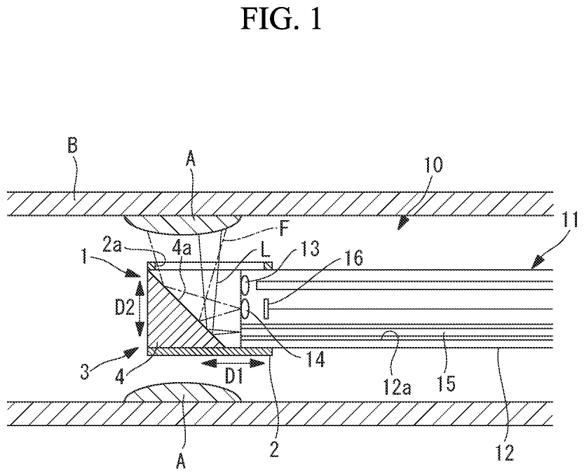

FIG. 1 shows a first configuration example of a therapeutic light irradiation unit 1 according to the present embodiment. The therapeutic light irradiation unit 1 is a cap to be attached to the distal end of an elongated insertion section 12 of an endoscope 11 as necessary. The therapeutic light irradiation unit 1 may be disposable.

The therapeutic light irradiation apparatus 10 includes an endoscope 11 and a therapeutic light irradiation unit 1.

The endoscope 11 is a front-view type and has, as well as an elongated insertion section 12, an illumination optical system (light irradiation section) 13 and an objective optical system 14 that are provided at the distal end part of the insertion section 12. The endoscope 11 also has a therapeutic light irradiation section (light irradiation section) 15 that emits therapeutic light L. A therapeutic light irradiation section 15 is, for example, an optical fiber probe that is inserted into a channel (lumen) 12a of the insertion section 12 and emits therapeutic light L from a distal end thereof.

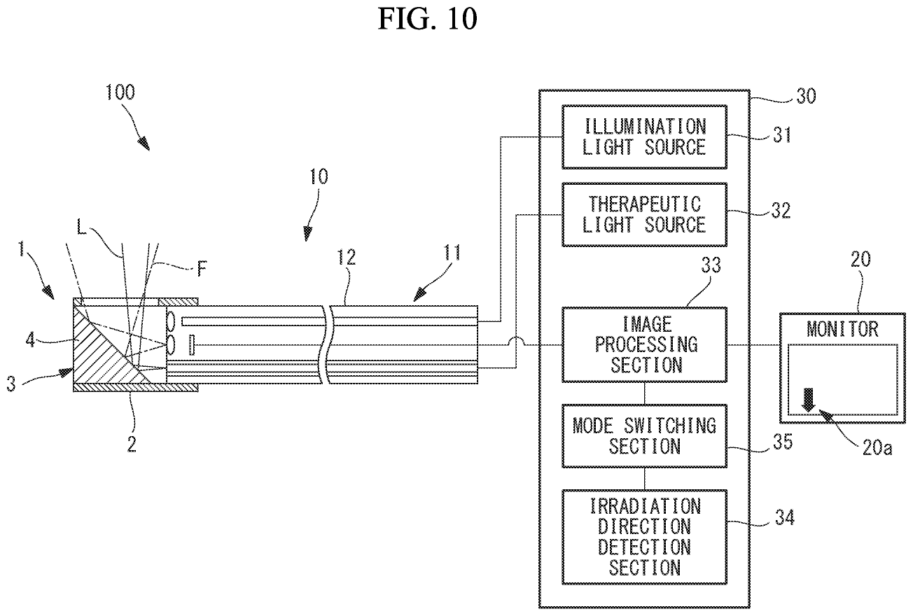

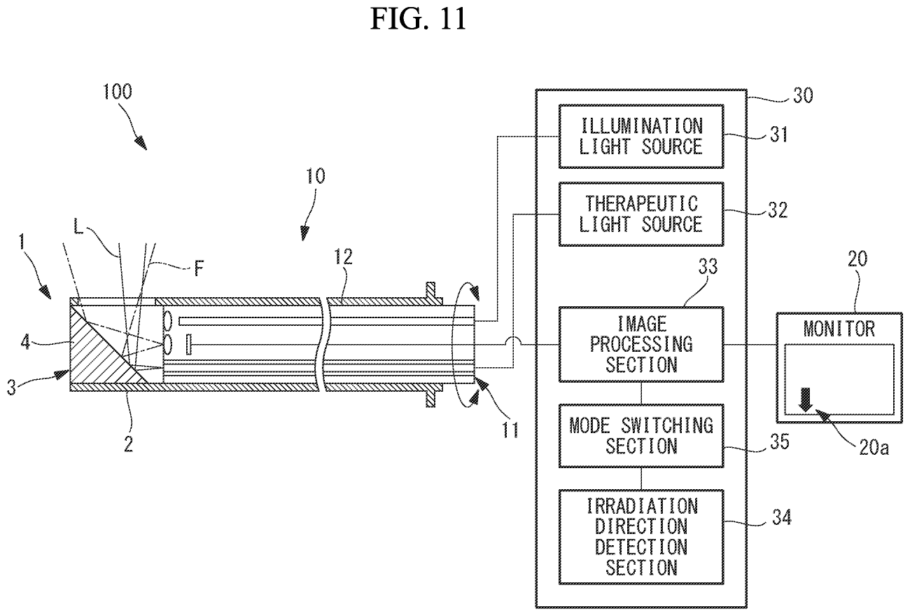

An illumination optical system 13 and the therapeutic light irradiation section 15 emit illumination light and therapeutic light L, respectively, in a first direction D1 along the longitudinal axis of the insertion section 12. The objective optical system 14 receives observation light incident from an object in the first direction D1 and observes the field of view F in the first direction D1. The field of view F observed by the objective optical system 14 is imaged by an image sensor 16, and the image of the field of view F is displayed on a display section 20 (see FIG. 10).

The therapeutic light irradiation unit 1 includes a connection section 2 to be attached to the distal end of the insertion section 12, and an irradiation direction switching section 3 for switching the irradiation direction of the illumination light and the therapeutic light L.

The connection section 2 is a tubular member that is open at least on the proximal end surface. The distal end part of the insertion section 12 is inserted into the connection section 2 from the proximal end side, so that the connection section 2 is detachably attached to the distal end part of the insertion section 12.

The irradiation direction switching section 3 includes a reflecting member 4 having a reflecting surface 4a. The reflecting member 4 is disposed on the front side of the illumination optical system 13, the objective optical system 14 and the therapeutic light irradiation section 15. The reflecting surface 4a faces the distal end surface of the insertion section 12 and is inclined with respect to the longitudinal axis of the insertion section 12. The reflecting member 4 reflects all or substantially all of each of the illumination light and the therapeutic light L in a second direction D2 on the reflecting surface 4a, and reflects the observation light, incident in the second direction D2, in a first direction D1. Thereby, the reflecting member 4 switches the irradiation direction of the illumination light and the therapeutic light L from the first direction D1 to the second direction D2, and switches the observation direction of the objective optical system 14 from the first direction D1 to the second direction D2. The second direction D2 is a direction intersecting the longitudinal axis of the insertion section 12.

The connection section 2 may be entirely made of a transparent material or may have a window 2a so that the illumination light, the therapeutic light L, and the observation light can travel in the second direction D2 between the inside and the outside of the connection section 2. The window 2a is formed by a hole or made of an optically transparent material.

Such a therapeutic light irradiation unit 1 is attached to the distal end of the insertion section 12 in a case in which the affected area A to be treated is located on the lateral side of the insertion section 12, in particular, in a case in which the affected area A is located in the lumen B and on the side wall as shown in FIG. 1. Thereby, the affected area A on the lateral side of the insertion section 12 can be irradiated with illumination light from the illumination optical system 13 and can be observed by the objective optical system 14. In addition, the lateral side affected area A can be irradiated with the therapeutic light L from the therapeutic light irradiation section 15 to be effectively treated. In this manner, the treatment target A on the lateral side can be captured in the image, and can be reliably irradiated with the therapeutic light L.

Also, in a state in which the therapeutic light irradiation unit 1 is not attached to the distal end of the insertion section 12, the affected area A on the front side of the insertion section 12 can be observed and treated. Therefore, attachment and detachment of the therapeutic light irradiation unit 1 enables both the light therapy for the treatment target A on the lateral side and the light therapy for the treatment target A on the front side.

In a state in which the therapeutic light irradiation unit 1 is attached to the distal end of the insertion section 12, the object in the image acquired by the image sensor 16 is a mirror image. Therefore, when the therapeutic light irradiation unit 1 is attached to the distal end of the insertion section 12, a mirror image correction mode for correcting a mirror image to a real image may be selected and executed. In the mirror image correction mode, the image corrected to a real image is displayed on the display section 20. The operator may be allowed to select the mirror image correction mode.

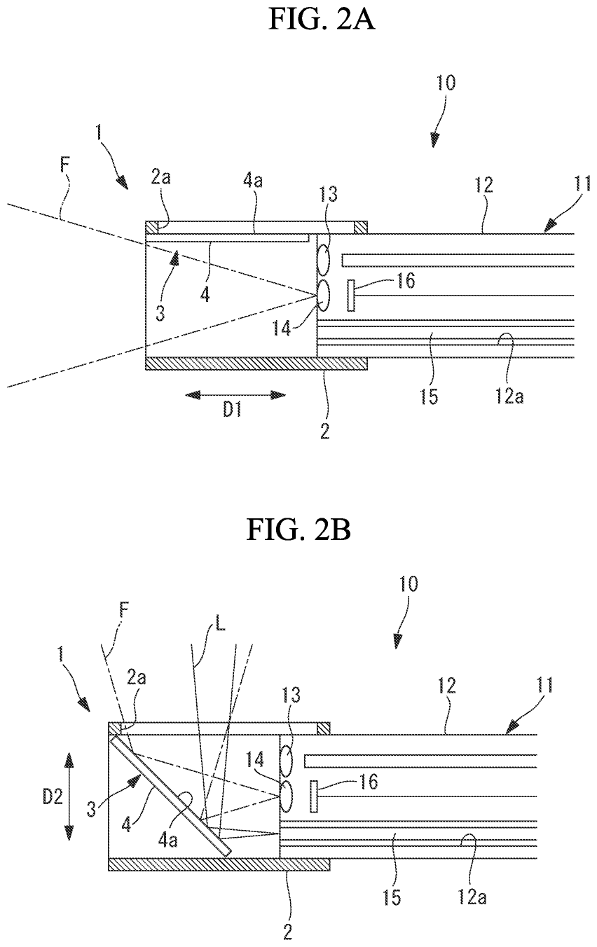

FIGS. 2A and 2B show a second configuration example of the therapeutic light irradiation unit 1 according to the present embodiment. In the second configuration example, the reflecting member 4 is a thin flat plate member that is rotatable between a first angle for front view shown in FIG. 2A and a second angle for side view shown in FIG. 2B.

The reflecting member 4 at the first angle is located at a position out of the front side of the optical systems 13 and 14 and the therapeutic light irradiation section 15. In FIG. 2A, the reflecting member 4 at the first angle is located radially outside the optical system 13 and parallel to the first direction D1.

The reflecting member 4 at the second angle is located on the front side of the optical systems 13 and 14 and the therapeutic light irradiation section 15, like the reflecting member 4 in the first configuration example. At the second angle, the reflecting surface 4a faces the distal end surface of the insertion section 12 and is inclined with respect to the longitudinal axis of the insertion section 12.

The reflecting member 4 is rotatable about an axis of rotation between a first angle and a second angle with one end thereof as the rotation center. The axis of rotation extends in a direction orthogonal to the longitudinal axis of the insertion section 12 and intersecting the first direction D1 and the second direction D2 (in the referred drawing, the direction perpendicular to the paper surface). Rotation of the reflecting member 4 from the first angle to the second angle switches the illumination direction and the observation direction from the first direction D1 to the second direction D2.

A user such as an operator positions the reflecting member 4 at the first angle when inserting the insertion section 12 into the body cavity. This allows the user to advance the insertion section 12 to a region where the target affected area A may be located while observing the front-view image in the field of view F on the front side of the insertion section 12. Moreover, the user positions the reflecting member 4 at the second angle when the user treats the affected area A on the lateral side. This allows the user to observe a side-view image in the field of view F on the lateral side of the insertion section 12 and irradiate the affected area A with the therapeutic light L when the affected area A on the lateral side is located substantially in the center of the field of view.

Thus, the user can observe and treat the treatment target A both on the front side and lateral side with the therapeutic light irradiation unit 1 attached to the distal end of the insertion section 12.

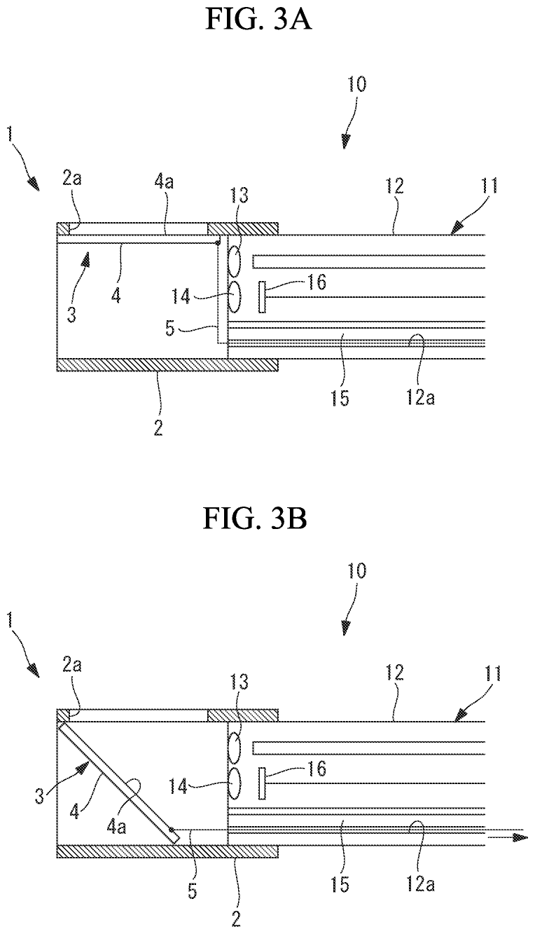

The reflecting member 4 is mechanically rotated between the first angle and the second angle. FIGS. 3A and 3B show an example of a mechanism for rotating the reflecting member 4. In this example, the irradiation direction switching section 3 further has a wire (operation member) 5 connected to the reflecting member 4.

The wire 5 is longer than the channel 12a, is disposed from the distal end side to the proximal end side of the insertion section 12 through the inside of the channel 12a, and has the distal end connected to the reflecting member 4.

When the therapeutic light irradiation unit 1 is attached to the distal end of the insertion section 12, the wire 5 is inserted into the channel 12a until the wire 5 protrudes from the proximal end of the insertion section 12, and the proximal end part of the wire 5 is exposed outside the channel 12a to a length that can be pulled by an operation unit (not shown) or manually. Here, in a state in which the therapeutic light irradiation unit 1 is attached to the distal end of the insertion section 12, as illustrated, the therapeutic light irradiation unit 1 is circumferentially positioned with respect to the insertion section 12 by the connection section 2 so that the channel 12a and the window 2a are positioned on opposite sides in the radial direction. Further, the distal end part of the wire 5 disposed inside the connection section 2 has a shape that curves toward the window 2a, and can be elastically deformed into a linear shape in the longitudinal direction of the channel 12a. Thus, the wire 5, which is pulled toward the proximal end side, rotates the reflecting member 4 from the first angle to the second angle. The wire 5, which is pressed toward the distal end side, rotates the reflecting member 4 from the second angle to the first angle.

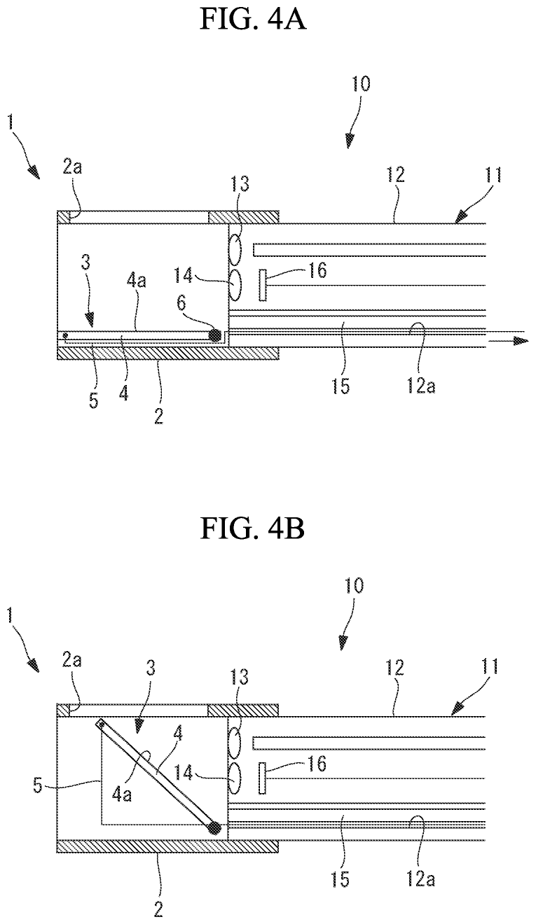

FIGS. 4A and 4B show another example of a mechanism for rotating the reflecting member 4. In this example, the reflecting member 4 is biased to a second angle by a spring 6 and is rotated from the second angle to the first angle with the wire 5 being pulled.

The operation member is not limited to the wire 5, and may be any member that can apply a force to the reflecting member 4 to rotate it about the axis of rotation.

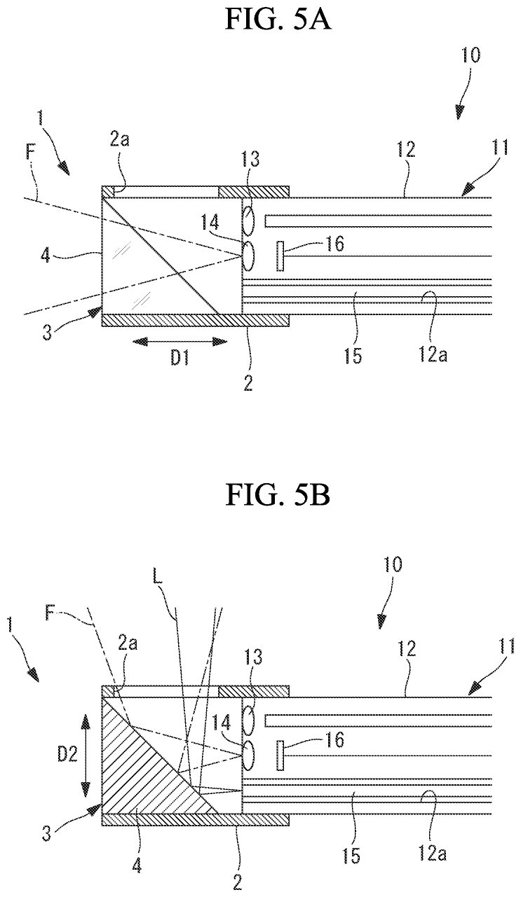

As shown in FIGS. 5A and 5B, the irradiation direction may be switched between the first direction D1 and the second direction D2 by changing the transmittance/reflectivity of the reflecting member 4, instead of rotating the reflecting member 4. FIG. 5A shows a state in which the reflecting member 4 has a low reflectivity (or a high transmittance), and FIG. 5B shows a state in which the reflecting member 4 has a high reflectivity (or a low transmittance).

In this case, a reflecting member 4 to be used has a transmittance and a reflectivity that change in response to electricity, light, or heat. For example, the reflecting member 4 to be used may be an optical plate containing an electrochromic crystal to electrically switch between transmission and reflection of light.

Another configuration may be such that the reflecting member 4: is made of a material that is a substance whose transmittance changes with light or heat; is irradiated with laser beam to increase its reflectivity; and then irradiates the affected area A on the lateral side with the therapeutic light L.

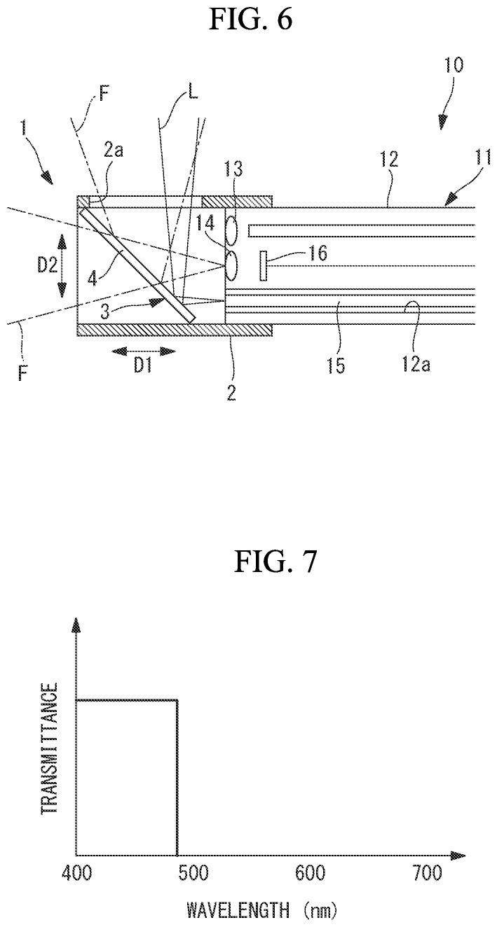

FIG. 6 shows a third configuration example of the therapeutic light irradiation unit 1 according to the present embodiment. In the third configuration example, the reflecting member 4 transmits part of the light in the first direction D1 and reflects the other part of the light in the second direction D2.

For example, the reflecting member 4 switches the irradiation direction of the illumination light and the therapeutic light L between the first direction D1 and the second direction D2 depending on the wavelength. In one example, as shown in FIG. 7, the reflecting member 4 is a dichroic mirror that transmits light in the blue band and reflects light that includes the therapeutic light L and has a longer wavelength than the blue band. In this case, the front-view image may be generated from the blue pixels of the image sensor 16 and the side-view image may be generated from the green and red pixels of the image sensor 16.

In a case in which the endoscope 11 acquires images by a frame sequential method in which an object is sequentially irradiated with three colors of red, green, and blue light as the illumination light, the side-view image may be generated in synchronization with the red and green light irradiation, and the front-view image may be generated in synchronization with the blue light irradiation.

The side-view image and the front-view image may be displayed in parallel on the display section 20, or may be alternatively displayed.

A user such as an operator can observe front-view images when inserting the insertion section 12 into the body, and can observe side-view images during treatment.

The reflecting member 4 may be a half mirror. In this case, half of each of the illumination light and the therapeutic light L is transmitted through the reflecting member 4 in the first direction D1, and the remaining half of each of the illumination light and therapeutic light L is reflected by the reflecting member 4 in the second direction D2.

In each of the above configuration examples, the position of the distal end of the insertion section 12 may be changeable within the connection section 2 in the first direction D1. For example, a stepped structure may be formed on the inner peripheral surface of the connection section 2 so that the position of the distal end of the insertion section 12 can be changed in multiple steps. Change in the position of the distal end of the insertion section 12 changes the distance in the first direction D1 from the distal end of the insertion section 12 to the reflecting member 4. This can change the observation position and the irradiation position of the therapeutic light L in the first direction D1.

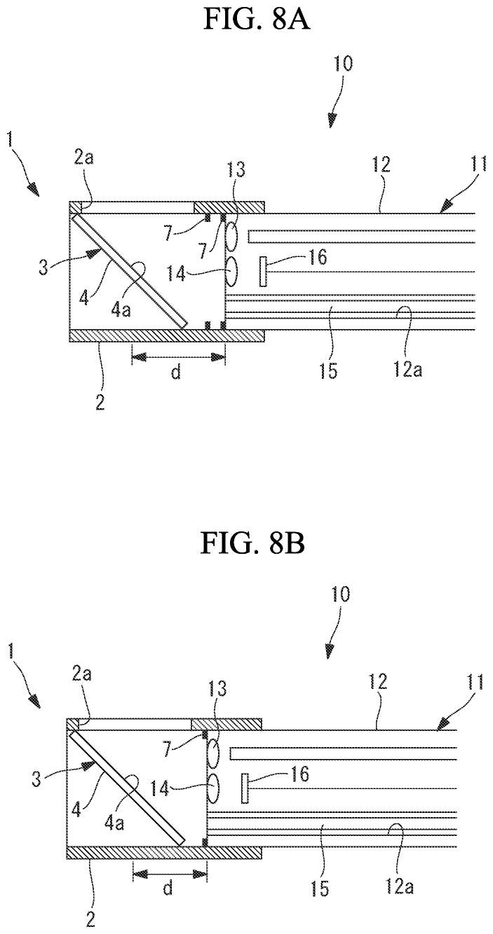

As shown in FIGS. 8A and 8B, the connection section 2 may have an inner peripheral surface provided with a plurality of projections (positioning portions) 7 projecting radially inward. The plurality of projections 7 are provided at a plurality of positions in the first direction D1.

The distal end surface of the insertion section 12 is abutted against the projection 7, and thereby the insertion section 12 is positioned. Moreover, the projection 7 has elasticity, allowing the insertion section 12 to be inserted to a deeper position when the insertion section 12 is pushed in to bend the projection 7.

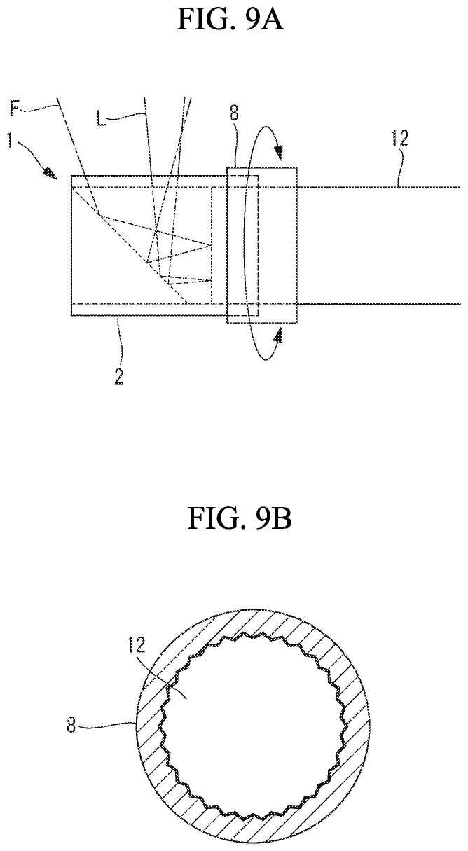

In each of the above configuration examples, as shown in FIG. 9A, the therapeutic light irradiation unit 1 may further include a fixture 8 that fixes the connection section 2 to the insertion section 12 at an arbitrary rotation angle about the longitudinal axis of the insertion section 12.

For example, the fixture 8 is a ring-shaped member that is fixed to the proximal end part of the connection section 2 and disposed on the outer peripheral surface of the distal end part of the insertion section 12. As shown in FIG. 9B, the fixture 8 has an inner peripheral surface having a plurality of grooves arranged in the circumferential direction, and the distal end part of the insertion section 12 has an outer peripheral surface having a plurality of projections that fit into the plurality of grooves of the fixture 8.

The user can integrally rotate the fixture 8 and the connection section 2 with respect to the insertion section 12, to rotationally position the fixture 8 and the connection section 2 with respect to the insertion section 12 at any position where the grooves fit into the projections.

The connection section 2 is fixed to the insertion section 12 at a desired rotation angle outside the body, and then the insertion section 12 is inserted into the lumen B. This makes it possible to easily change the position of the affected area A to be subjected to light therapy within the lumen B.

FIG. 10 shows a configuration example of a therapeutic light irradiation system 100 including a therapeutic light irradiation unit 1.

The therapeutic light irradiation system 100 includes a therapeutic light irradiation unit 1, an endoscope 11, a display section 20 and a processor 30. A proximal end of the insertion section 12 is connected to the processor 30. The processor 30 includes: an illumination light source 31 that supplies illumination light to the illumination optical system 13; a therapeutic light source 32 that supplies therapeutic light L to the therapeutic light irradiation section 15; and an image processing section 33 that processes an image acquired by the image sensor 16. The correction of the mirror image may be performed by the image processing section 33.

The therapeutic light irradiation system 100 may further include an irradiation direction detection section 34 that detects the irradiation directions of the illumination light and the therapeutic light L.

When the irradiation direction is detected to be the second direction D2, the mode switching section 35 may automatically switch to the mirror image correction mode. Alternatively, an indication 20a indicating the detected irradiation direction may be displayed on the display section 20, and the user may execute the mirror image correction mode based on the indication 20a.

The irradiation direction detection section 34 may detect whether the therapeutic light irradiation unit 1 is attached to the distal end of the insertion section 12. In the case of the reflecting member 4 of the first configuration example, it is possible to detect whether the irradiation direction is the first direction D1 or the second direction D2 based on the presence or absence of the therapeutic light irradiation unit 1.

In the case in which the movable reflecting member 4 of the second configuration example is provided, the irradiation direction detection section 34 may have a sensor that detects the rotation angle (first angle or second angle) of the reflecting member 4. The sensor may detect the rotation of the reflecting member 4 based on the movement amount of the wire 5.

In each of the above configuration examples, the connection section 2 is a cap to be attached to the distal end of the insertion section 12. Alternatively, as shown in FIG. 11, the connection section 2 may be a tubular sheath that covers the insertion section 12 in the longitudinal direction. Rotation of the connection section 2 about the longitudinal axis with respect to the insertion section 12 can change the irradiation position and the observation position in the circumferential direction about the longitudinal axis.

The image may be three-dimensionally displayed on the display section 20 according to the rotation angle of the connection section 2. As a result, the entire periphery of the lumen B can be observed, and can be irradiated with the therapeutic light L.

In each of the above configuration examples, the therapeutic light irradiation unit 1 may be provided as part of the endoscope 11. That is, the endoscope 11 may include, at the distal end of the insertion section 12, the illumination optical system 13, the therapeutic light irradiation section 15, and the irradiation direction switching section 3. The irradiation direction switching section 3 may be detachably attached to the distal end of the insertion section 12 or may be integrated in the insertion section 12.

The above examples describe cases in which the present disclosure is applied to PIT, but the following applications are also possible. In other words, the present disclosure can be applied not only to PIT but also to medical equipment using observation light and therapeutic light, such as PDT that performs light therapy while monitoring fluctuations in the intensity of light such as fluorescence similarly to PIT. In addition, the present disclosure can also be applied to a light therapy probe that exclusively irradiates therapeutic light. In this case, the therapeutic light irradiation unit, the therapeutic light irradiation apparatus, and the therapeutic light irradiation system of the present disclosure may be used as a device separate from the endoscope.

What is claimed is:

1. A therapeutic light irradiation device comprising:
a reflecting surface disposed on a path of a therapeutic light emitted in a first direction from an insertion section of an endoscope, and configured to switch an irradiation direction of the therapeutic light between the first direction and a second direction intersecting the first direction; and
a connector configured to detachably connect the reflecting surface to the insertion section.

2. The therapeutic light irradiation device according to claim 1, further comprising:
a rotation mechanism configured to rotate the reflecting surface about an axis of rotation between a first position where the reflecting surface is disposed on the path of the therapeutic light emitted in the first direction and a second position where the reflecting surface is not disposed on the path of the therapeutic light emitted in the first direction.

3. The therapeutic light irradiation device according to claim 2, further comprising:
an operation member configured to operate the rotation mechanism to rotate the reflecting surface about the axis of rotation.

4. The therapeutic light irradiation device according to claim 3,
wherein the operation member comprises a wire configured to transmit a force along the insertion section to operate the rotation mechanism to rotate the reflecting surface about the axis of rotation.

5. The therapeutic light irradiation device according to claim 1,
wherein the reflecting surface is configured to switch the irradiation direction of the therapeutic light having a first wavelength between the first direction and the second direction and to transmit a light having a second wavelength.

6. The therapeutic light irradiation device according to claim 1,
wherein the connector is configured to be detachably connected to the insertion section at different positions to change a relative position of the reflecting surface relative to the insertion section.

7. A therapeutic light irradiation apparatus comprising:
an insertion section;
a light source configured to radiate light in a first direction from the insertion section; and
a reflecting surface disposed on a path of the light emitted in the first direction from the insertion section, and configured to switch an irradiation direction of the light between the first direction and a second direction intersecting the first direction.

8. The therapeutic light irradiation apparatus according to claim 7, further comprising:
a rotation mechanism configured to rotate the reflecting surface about an axis of rotation between a first position where the reflecting surface is disposed on the path of the light emitted in the first direction and a second position where the reflecting surface is not disposed on the path of the light emitted in the first direction.

9. The therapeutic light irradiation apparatus according to claim 8, further comprising:
a wire configured to transmit a force along the insertion section to operate the rotation mechanism to rotate the reflecting surface about the axis of rotation.

10. The therapeutic light irradiation apparatus according to claim 7, wherein:
the light emitted in the first direction has a wavelength within a wavelength range; and
the reflecting surface is configured to switch the irradiation direction of the light between the first direction and the second direction based on the wavelength of the light being in the wavelength range, and to transmit another light having a wavelength not within the wavelength range.

11. An endoscope comprising:
an insertion section;
an illumination light source, arranged in the insertion section, configured to radiate illumination light in a first direction;
a therapeutic light source, arranged in the insertion section, configured to radiate therapeutic light in the first direction;
a reflecting surface disposed on a path of the illumination light and the therapeutic light emitted in the first direction from the insertion section, and configured to switch irradiation directions of both the illumination light and the therapeutic light between the first direction and a second direction intersecting the first direction; and
a connector configured to detachably connect the reflecting surface to the insertion section.

12. The endoscope according to claim 11, further comprising:
a rotation mechanism configured to rotate the reflecting surface about an axis of rotation between a first position where the reflecting surface is disposed on the path of the therapeutic light and the illumination light emitted in the first direction and a second position where the reflecting surface is not disposed on the path of the therapeutic light and the illumination light emitted in the first direction; and
a wire configured to transmit a force along the insertion section to operate the rotation mechanism to rotate the reflecting surface about the axis of rotation.

13. The therapeutic light irradiation device according to claim 1,
wherein the first direction is along a longitudinal axis of the insertion section.

14. The therapeutic light irradiation device according to claim 4, wherein:
the wire is longer than a lumen of the endoscope;
a distal end of the wire is connected to the rotation mechanism; and
a proximal end of the wire protrudes from a proximal end of the lumen.

15. The therapeutic light irradiation device according to claim 1,
wherein a portion of the connector disposed in the second direction comprises an optically transparent material or the connector defines a hole disposed in the second direction so that the therapeutic light can travel in the second direction between an inside and an outside of the connector.
